(19) United States
(12) Patent Application Publication
(10) Pub. No.: US 2009/0105610 A1
(43) Pub. Date: Apr. 23, 2009

(54) SYSTEMS AND METHODS FOR COLLECTION OF CELL CLUSTERS

(75) Inventors: Peter GOMBRICH, Novato, CA (US); Eugene DIMONTE, Aurora, IL (US); Edward EATON, Eola, IL (US); Eric LARSON, Pecatonica, IL (US)

Correspondence Address: HAMRE, SCHUMANN, MUELLER & LARSON, P.C., P.O. BOX 2902, MINNEAPOLIS, MN, 55402-0902, US

(73) Assignee: DIAMICS, INC., Novato, CA (US)

(21) Appl. No.: 12/339,870

(22) Filed: Dec. 19, 2008

Related U.S. Application Data
(63) Continuation of application No. 11/318,025, filed on Dec. 23, 2005.
(60) Provisional application No. 60/642,008, filed on Jan. 6, 2005.
(60) Provisional application No. 60/681,901, filed on May 17, 2005.
(60) Provisional application No. 60/686,150, filed on Jun. 1, 2005.
(60) Provisional application No. 60/708,150, filed on Aug. 15, 2005.
(60) Provisional application No. 60/729,854, filed on Oct. 25, 2005.
(60) Provisional application No. 60/729,857, filed on Oct. 25, 2005.

(51) Int. Cl. A61B 10/00

(52) U.S. Cl. 600/572

(57) ABSTRACT

A cell collector and cell collection method are provided for collecting clusters of cells for subsequent analysis of the cells to screen for abnormalities. The cell collector is designed to enhance the capability of the collector to pick-up clusters or clumps of cells, and to facilitate transfer of the collected clusters of cells onto a receiving structure, for example a slide. In one embodiment, a combination of the material of the collector, the texture of the collection surface of the collector, and the use of expansion and rotation of the collector during collection facilitate the collection of the clusters of cells.

This application claims the benefit of U.S. Provisional Application No. 60/642,008 filed Jan. 6, 2005; U.S. Provisional Application No. 60/681,901 filed May 17, 2005; U.S. Provisional Application No. 60/686,150 filed Jun. 1, 2005; U.S. Provisional Application No. 60/708,150 filed Aug. 15, 2005; U.S. Provisional Application No. 60/729,854 filed Oct. 25, 2005; and U.S. Provisional Application No. 60/729,857 filed Oct. 25, 2005 and claims priority to U.S. application Ser. No. 11/318,025 filed Dec. 23, 2005. Each of the above-referenced applications is incorporated herein by reference in their entirety.

### TECHNICAL FIELD

This disclosure relates generally to the collection of cell clusters for later use in examining the cell clusters. More specifically, this disclosure relates to a collector that is designed to enhance the capability of the collector to pick-up clusters or clumps of cells, for example from a cervix, and where the clusters of cells are collected in a manner where the spatial arrangement of the collected clusters of cells is preserved.

### BACKGROUND

It is often necessary to collect various cell samples from patients for the purposes of screening for, detecting, and ultimate treatment of, a number of diseases and abnormalities. One of the major reasons for the collection of cellular samples is for the purpose of screening patients for cancer. For example, urine, sputum, breast nipple and fine needle aspirates, and exfoliated cells of the uterine cervix are screened by cytotechnicians and pathologists for the presence of abnormal cells suggestive of the presence of a solid tumor. When such suspicious cells are found, a more definitive diagnosis is reached by removing a sample of the tissue where a lesion is suspected, and submitting the sample for review by a pathologist.

It is generally accepted that diagnosis of cancer at its earliest stages affords the greatest opportunity for effective treatment. A corollary to this is that early diagnosis of a solid tumor corresponds to recognition of localized abnormalities, which at the cellular level are not that different from the surrounding tissue. This presents a challenge for screening of cellular samples where all context and comparison to neighboring cells is lost. One approach to this problem is to concentrate upon elements, i.e. groups of cells, which more closely approximate intact tissue elements. In fact, the presence of such clusters of cells, in and of itself, can be considered to be suggestive of a pre-cancerous or cancerous condition. However, it is also the case that normal tissue elements can be represented as cell clusters in samples collected for cytologic analysis.

Conventional sampling methods utilized in current screening procedures are capable of acquiring cells from a lesion, but then often disperse these cells into a typically much larger number of normal cells obtained from outside of the boundaries of the lesion. This dispersion results in the evaluation of a sample being an exercise in the detection of a rare event; that is, finding one or a few abnormal cells within a background consisting of a very large number (e.g. 50,000-300,000) of normal cells. Furthermore, and perhaps most significantly, dispersion eliminates the information that can be gained from determining the biological characteristics of small areas that might represent preneoplastic lesions. This essential information is present in the relationship among cells, and is not apparent by examining individual cells in isolation from adjacent cells within a tissue. Dispersion also precludes using the sample to determine the location of the lesion on the patient.

Therefore, it would also be desirable to provide improved cell collection procedures that facilitate the collection of cell clusters and which retain the spatial relationship that existed between cells prior to collection.

### SUMMARY

A cell collector and cell collection method are provided for collecting clusters of cells for subsequent analysis of the cells to screen for abnormalities. The cell collector is designed to enhance the capability of the collector to maintain the integrity of cellular clusters or clumps, and to facilitate transfer of the collected clusters of cells onto a receiving structure, for example a slide. In one embodiment, a combination of the material of the collector, the texture of the collection surface of the collector, and the use of expansion and rotation of the collector during collection facilitate the collection of the clusters of cells.

Preferably, clusters of cells are transferred from the collector to the receiving structure in such a way as to retain the spatial relationships that existed between the cells in the clusters prior to sampling. Orientation marks on the collector and the receiving structure assist in maintaining the spatial relationship during transfer.

The collector is expanded during collection as well as during transfer of the cells. Expansion during collection and transfer can occur through the use of air, by a mechanical expansion system, or through a combination of air and a mechanical system. Preferably, the collector can be expanded during transfer such that the cell clusters obtained from the endo- and ecto-cervical regions end up on a generally common plane for subsequent transfer to the receiving structure.

Cell cluster collection can be applied to a number of regions of the body, for example the cervix, the bladder, the lungs, the colon, and the ovaries. The clusters of cells can be collected from tissue, urine, induced sputum, cells washed from ovaries, and the like.

### BRIEF DESCRIPTION OF FIGURES

FIGS. 1A-C illustrate an example of cell collection from a uterine cervix.

FIGS. 2A and 2B are a side view and a cross sectional view taken along line A-A, respectively, of one embodiment of a cell collector assembly according to the present invention.

FIG. 2C is a detailed view of the expandable collection tip of the cell collector assembly.

FIG. 3 is a cross-sectional view of the cell collector attached to a collector handle assembly.

FIG. 4 is a schematic diagram of a user's hand holding the collector handle assembly.

FIGS. 5A-C are cross sectional views of the tip of the cell collector illustrating expansion of the cell collection tip during cell cluster collection.

FIGS. 6A-C illustrate the steps of cell cluster collection from a cervix using the cell collector.

FIGS. 7A-C illustrate a collector handle assembly that can rotate the cell collector during collection.

FIGS. 8A-B illustrate the tip of the cell collector prior to and after inflation, respectively, but prior to transfer, with colored marker simulating collected clusters of cells.

FIG. 9 illustrates another embodiment of collector handle assembly.

FIG. 10 illustrates another embodiment of a cell collector and collector handle assembly.

FIGS. 11A-C are detailed views of the tip of the cell collector of FIG. 10 illustrating how expansion and rotation during collection occurs.

### DETAILED DESCRIPTION

A collector that is constructed to enhance the ability of the collector to pick-up clusters or clumps of cells, and to facilitate transfer of collected clusters of cells onto a receiving structure, for example a slide. In one embodiment, a combination of the material of the collector, the texture of the collection surface of the collector, and the use of expansion and rotation of the collector during collection facilitate the collection of cell clusters. Collected clusters of cells can then be transferred from the collector to the receiving structure in such a way as to retain the spatial relationships that existed between the cells in the clusters prior to sampling. Orientation marks on the collector and the receiving structure assist in maintaining the spatial relationship during transfer.

For purposes of explanation, the inventive concepts will be discussed below with respect to the collection of clusters of cells from a cervix to screen for cervical cancer. However, it is to be realized that the inventive concepts can be used to collect cell clusters from other regions of the body for use in screening for other diseases, for example the bladder to screen for bladder cancer, the lungs to screen for lung cancer, breasts to screen for breast cancer, the colon to screen for colon cancer, and the ovaries to screen for ovarian cancer. The clusters of cells can be collected from tissue, urine, induced sputum, breast secretions, cells washed from ovaries, and the like.

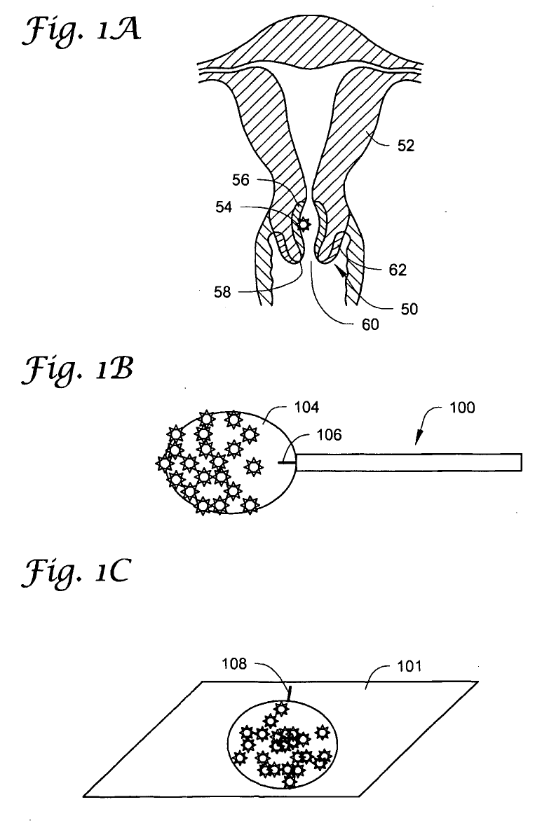

FIGS. 1A-C illustrate the concepts of cell cluster collection from a uterine cervix 50. FIG. 1A illustrates the cervix 50 formed by a uterus 52, with the cervix including a cervical canal 60, an endocervix 56, an ectocervix 62, and a transition zone 58 illustrated by shading that extends from the ectocervix to the endocervix. An exemplary lesion 54 is illustrated in the transition zone 58 at the endocervix 56 of the cervix.

FIG. 1B illustrates the concepts of a cell collector 100 that can be used to collect cells and cell clusters from the cervix 50. The collector 100 has a surface 104 that can conform to the contours of the cervix and which has properties such that clusters of cells from both the ecto- and endocervices 62, 56 are collected by the surface 104 to ensure collection of cell clusters from the transition zone 58, while preserving the spatial relationships among the collected cell clusters.

In addition, the collector 100 has a visible orientation mark 106 to permit the individual collecting the clusters of cells to orient the collector upon sampling of the cervix, and maintain that orientation upon subsequent transfer of cell clusters to a receiving structure 101 which also includes a corresponding orientation mark 108 as shown in FIG. 1C. Cell clusters can be transferred to the receiving structure 101 by contacting the surface 104 with the receiving structure 101 which is configured so that cell clusters transfer to the structure 101 rather than remain adhered on the surface 104. During transfer, the orientation marks 106, 108 are aligned, so that once transferred, cell clusters on the structure 101 have the same spatial relationship as they did on the collector 100. The cell clusters can then be analyzed to screen for potential abnormalities.

The cell collector 100 can have a number of different configurations as long as it is capable of collecting clusters of cells from both the endo- and ectocervices 56, 62 to ensure collection of cell clusters from the transition zone 58. In one embodiment, a combination of the material of the collector surface 104, the texture of the collector surface 104, and the use of expansion and rotation of the collector surface during collection facilitates the collection of the clusters of cells.

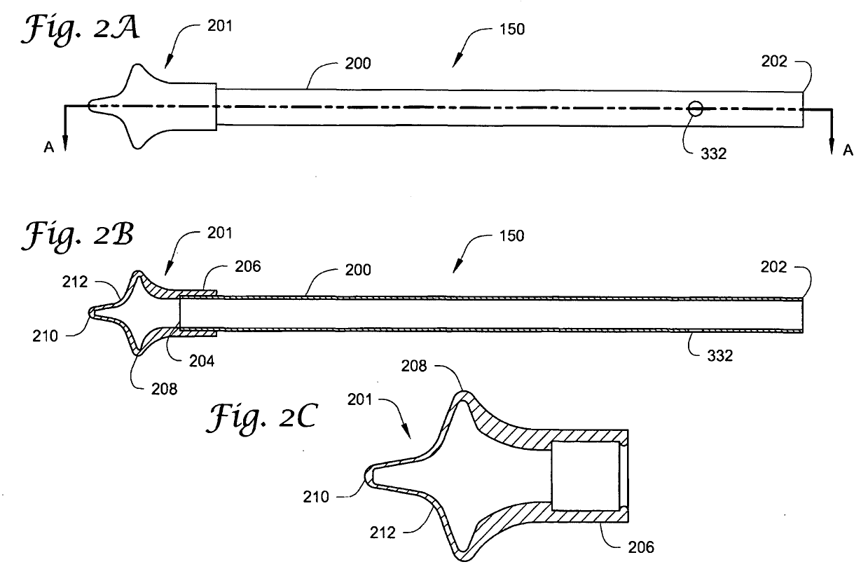

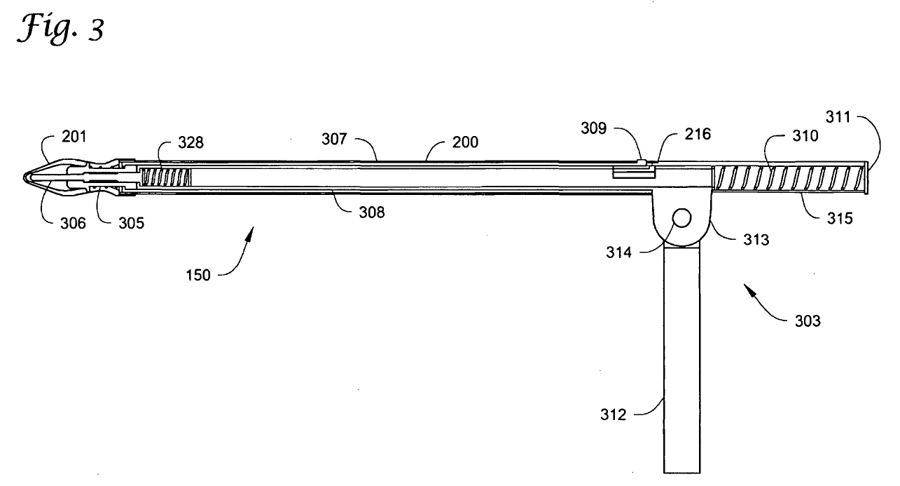

With reference now to FIGS. 2A-C, details of a cervical cell collector assembly 150 embodying the concepts of the invention are illustrated. The collector assembly 150 includes a hollow tube 200 that is detachably connected to an expandable collection tip 201. The tube 200 is made from, for example, plastic or cardboard. The expandable tip 201, which is also the cell collection region of the collector 150, is a resiliently flexible structure that is made of an elastomeric material, for example a thermoplastic elastomer alloy such as Versaflex® CL30 available from GLS Corporation of McHenry, Ill. The expandable tip 201 preferably has a texture that enhances the ability of the collector to collect clusters of cells from the transition zone 58 upon expansion and rotation of the tip 201. For example, the tip 201 can have a texture of MT-11010. Other elastomeric materials could be used for the tip 201, for example microporous polyvinyl acetate, nitrile rubber, nitrile foam, urethane foam, silicone rubber, latex rubber, polyurethane and other elastomers having low durometer, high percent elongation and adequate texture to enhance collection of cell clusters.

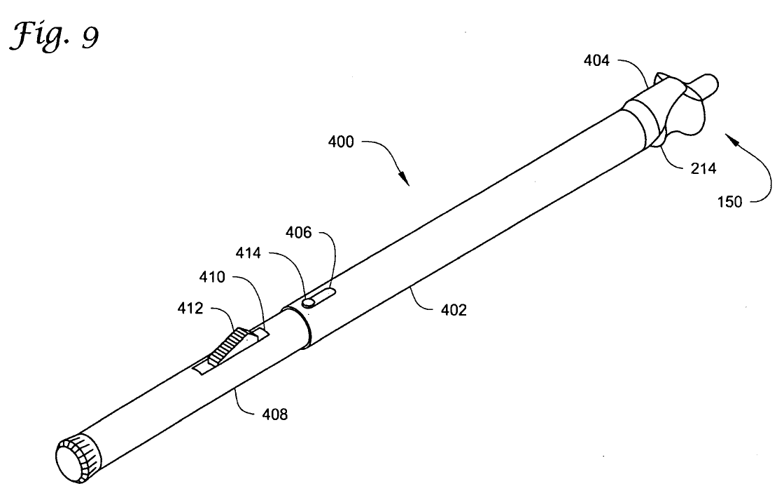

The tube 200 is generally hollow from one end 202 to the other end 204, with the end 202 of the tube 200 being open. With reference in particular to FIG. 2C, the expandable tip 201 in its as formed, original state includes a neck portion 206 detachably connected to the end 204 of the tube 200, a central enlarged shoulder 208, a tip region 210, and a transition section 212 extending between the shoulder 208 and the tip region 210. As shown in FIG. 9, an o-ring 214 can be provided around the neck portion 206 of the collection tip 201 to aid in retaining the tip 201 on the tube 200.

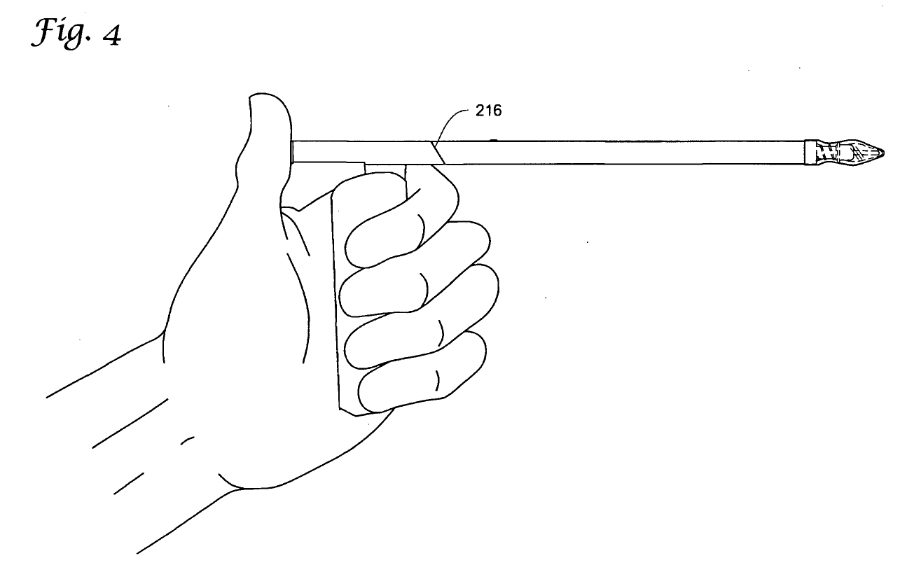

FIGS. 3-5 show the cell collector assembly disposed on a collector handle assembly 303 for use in taking a cell sample. The assembly 303 includes an inner casing 308 and an outer casing 307, with the tube 200 being disposed around the outer casing 307, and the outer casing 307 being slidably disposed on the inner casing 308. A probe 306 projects forwardly from inside the inner casing 308 into the interior of the expandable tip 201. An expander probe 305 is disposed at the end of the assembly 303 surrounding the probe 306, with an end 320 of the probe 305 disposed in the outer casing 307 at the end of the outer casing 308. An opposite end 322 of the probe is enlarged and includes a shoulder 324.

The probe 306 can have a diameter of approximately 2 mm and project beyond the end of the expander probe 305 a distance between approximately 8 to 10 mm. The body of the expander probe 305 forward of the shoulder 324 can have a diameter of approximately 6 mm, while the shoulder 324 has a diameter of approximately 10 mm.

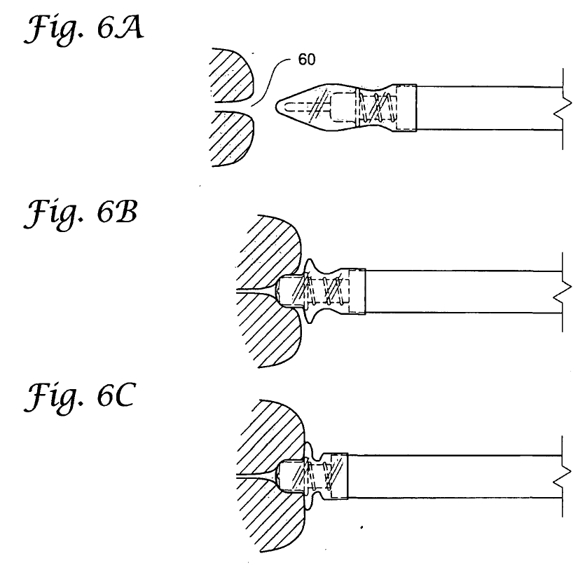

A coil spring 326 is disposed between the shoulder 324 and the end of the outer casing 307 for biasing the expander probe 305 to the left in FIGS. 3 and 5A-C. In addition, a coil spring 328 is disposed inside the inner casing 308 between the end of the probe 306 and a fixed ring 330 disposed in the inner casing. The spring 328 biases the probe 306 to the left in FIGS. 3 and 5A-C.

The outer tube 307 also includes a tube lock 309. The tube lock 309 comprises a resilient member fixed to the outer tube 307 that projects upwardly through an aperture 332 (see FIGS. 2A and 2B) formed in the tube 200 of the collector assembly 150. The tube lock 309 and aperture 332 cooperate to lock the tube 200 to the outer tube 307 of the handle assembly 303.

Returning to FIG. 3, a return spring 310 is disposed within the outer tube 307 between the end of the inner tube 308 and a spring cap 311 that is disposed at the end of the outer tube 307. The spring 310 biases the outer tube 307 toward the right in FIG. 3 while biasing the inner tube 308 toward the left, to return the outer 307 and inner tubes 308 to a home position shown in FIG. 3.

A handle 312 is fixed to a support 313 that is connected to the inner tube 308. The handle 312 is rotatably secured to the support 313 by a pivot 314 to allow the handle 312 to pivot between the position shown in FIG. 3 and a collapsed position where the handle 312 is generally parallel to the casings 307, 308. The outer tube 307 is formed with a slot 315 that allows relative sliding movements between the outer tube 307 and the support 313. The slot 315 extends to the right of the support 313 to the cap 311 in FIG. 3.

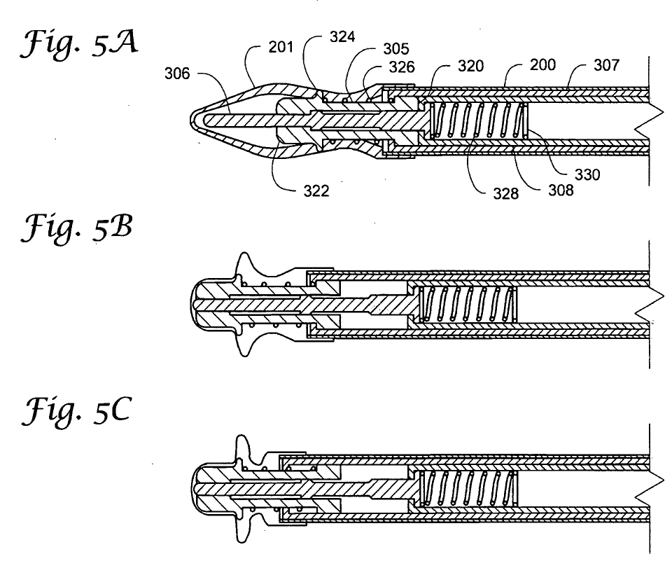

As best seen in FIGS. 3 and 4, the diameter of the outer tube 307 changes from a smaller diameter section that is designed to receive the tube 200 of the collector 150 to a larger diameter section adjacent the handle 213 and extending to the right of the support 313 in FIG. 3. The transition between the smaller diameter section and the larger diameter section forms a shoulder 216 (FIG. 4) against which the end of the tube 200 abuts. If desired, the end 202 of the tube 200 can be angled to match an angle formed by the shoulder 216. The angle of the shoulder 216 and the angle on the tube 200 can be aligned when the collector assembly 150 is slid onto the handle assembly 303 to help ensure that the collector assembly 150 is properly oriented on the handle assembly 303.

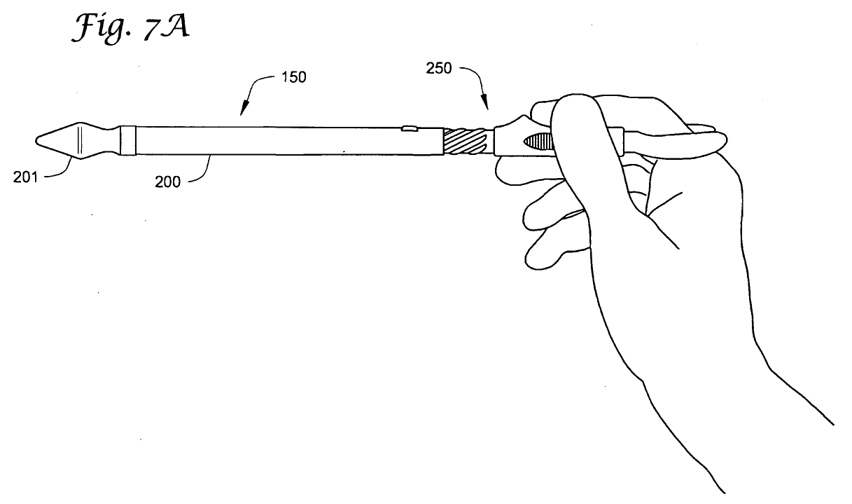

FIG. 4 is a schematic diagram of a hand holding onto the handle 312 with a thumb pressed against the spring cap 311. FIGS. 5A-C and FIGS. 6A-C, together with FIG. 4, show the process of collection using the cell collector assembly 150. The user initially inserts the cell collector assembly 150 onto the handle assembly 303. In doing so, the end of the probe 306 engages the tip region 210 of the expandable tip 201 causing the expandable tip to flatten out and temporarily reduce the shoulder 208 on the tip 201, as shown in FIGS. 5A and 6A. This improves the user's sight lines for inserting the collector into the cervix.

The user then pushes on the spring cap 311 with the thumb or other digit as shown in FIG. 4. This causes the outer casing 307 to be moved forward along with the expander probe 305, as shown in FIG. 5B. When the probe 305 moves forward, it causes the shoulder 208 of expandable tip 201 to expand outward from its flattened state, as shown in FIGS. 5B and 6B. The expander probe 305 bottoms out when it becomes flush with the end of the probe 306 after approximately 8 to 10 mm of travel, as shown in FIG. 5B. The expander probe 305 expands the endo-cervical canal to approximately 6 mm, with the expandable tip 201 in contact with the canal. Once the probe 305 bottoms out, continued pushing by the thumb continues movement of the outer casing 307 another approximately 3 to 4 mm, and at the same time pushes the tube 200 forward. As a result, the shoulder 208 and/or transition section 212 of the expandable tip 201 are compressed against the ectocervix 62 as shown in FIG. 6C.

During its movements, the expander probe 305 expands the tip region 210 of the expandable tip 201 into engagement with the endocervix 56. In addition, the shoulder 208 and/or transition section 212 of the expandable tip 201 compresses against the ecto-surface of the cervix 50. As a result, both endocervical and ectocervical cells, including cells from the transition zone 58, can be collected.

The expandable tip 201 is also rotated during collection in order to collect clusters of cells from the transition zone by shearing cell clusters from the transition zone 58 assisted by the texture of the tip 201. The tip 201 is rotated, for example, twenty to thirty degrees. The tip 201 can be rotated by the user manually rotating the handle assembly 303 and the collector assembly 150 connected thereto. Alternatively, the tip 201 can be rotated using a suitable mechanical rotation mechanism which causes rotation of the tip 201 once the tip region 210, shoulder 208 and transition section 212 of the tip 201 are expanded by the handle assembly 303 into contact with the endo- and ecto-cervices.

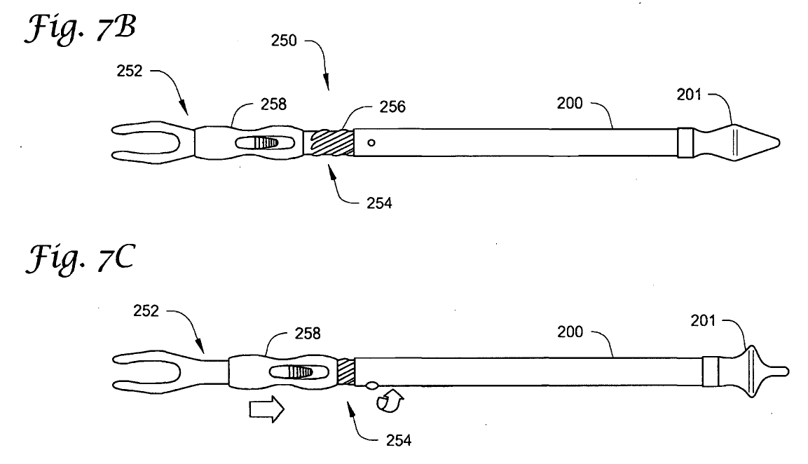

An example of a mechanical rotation mechanism is illustrated in FIGS. 7A-C. FIG. 7A illustrates the collector assembly 150 disposed on a handle assembly 250. The assembly 250 includes a U-shaped end portion 252, and an expansion and rotation portion 254 rotatably connected the U-shaped end portion 252 to permit rotation of the portion 254 relative to the end portion 252. The end of the portion 254 surrounded by the tip 201 is configured in a manner similar to that shown in FIGS. 5A-C. The opposite end of the portion 254 is provided with helical teeth 256 on the outer surface thereof.

A gripping sleeve 258 is slidably disposed on the portion 252 and the portion 254 over where the portions 252, 254 connect. Helical teeth (not shown) are disposed on the inside surface of the sleeve 258 for engagement with the teeth 256 on the portion 254.

During use of the assembly 250, after mounting the collector assembly 150 onto the handle assembly 250, as the user inserts the probe, the probe 305 (shown in FIGS. 5A-C) is moved forward, causing the tip 201 to expand (FIG. 5B). Continued pushing by the user causes the tip 201 to expand further to engage against the ecto-cervix (FIG. 5C). The engagement with the ecto-cervix prevents further insertion, and causes the gripping sleeve 258 to move forward in the direction of the arrow in FIG. 7C. The sleeve 258 eventually moves far enough to contact the helical teeth 256. Continued advancement of the sleeve 258 and the engagement of the helical teeth causes the portion 254 together with the collector 150 to rotate as shown by the arrow in FIG. 7C.

After insertion, and expansion and rotation to achieve cell cluster collection, the pressure is released and the return spring brings the mechanism back to the original position. The tube lock 309 is depressed and the cervical cell collector assembly 150 is then detached.

FIG. 9 shows another embodiment of a collector handle assembly 400 with the cell collector assembly 150 mounted thereon. The assembly 400 includes a front tube 402 having a deflector 404 connected thereto at the front end thereof. The handle assembly 400 is designed so that the tube 200 of the collector assembly 150 is slid into the tube 402 to mount the collector assembly 150. When the collector assembly 150 is mounted on the assembly 400, the deflector 404 flattens the shoulder 208 on the tip 201 to improve the sight lines for insertion during collection. The tube 402 also includes a slot 406 near the rear end thereof. The interior of the tube 402 around which the tube 200 is disposed is configured similarly as in FIGS. 5A-C.

The assembly 400 also includes a rear tube 408 having a front end thereof received within the rear end of the tube 402. A slot 410 is formed in the rear tube 408 and a button 412 is slideably disposed in the slot 410. The button 412 is connected to a projection 414 disposed within the slot 406 of the front tube 402.

The button 412 is illustrated in FIG. 9 at a home position, which is also the insertion position of the assembly 400. After properly inserted, the user pulls back on the button 412, and the button 412 moves to the end of the slot 410 to a rear button position. Since the button 412 is connected to the projection 414, the projection 414 also moves backward, which pulls the front tube 402 backward relative to the collector assembly 150 to release the deflection of the collection tip 201 caused by the deflector 404. Subsequently, the user pushes the button 412 forward to expand the collection tip 201. The button 412 is connected to the expansion mechanism shown in FIGS. 5A-C in such a manner that expansion occurs from the home position of the button to the forwardmost position of the button in the slot 410.

Once the button 412 is pushed all the way forwardly and the collection tip expanded, the tip is then rotated. The tip can be manually rotated, as discussed above, by manually rotating the rear tube 408. Alternatively, a suitable mechanical rotation mechanism can be provided for rotating the collection tip.

After collection, cell clusters can be transferred from the tip 201 to a receiving structure for subsequent analysis of the cell clusters. Examples of suitable receiving structures include a slide, a petri dish, and other structures to which cells may be transferred for subsequent analysis of the cell clusters. The surface of the receiving structure has greater adhesiveness than the surface of the tip 201 containing cell clusters to enhance the transfer of cell clusters from the tip to the receiving structure. When the receiving structure is a slide, the slide can be provided with a coating that results in the greater adhesiveness.

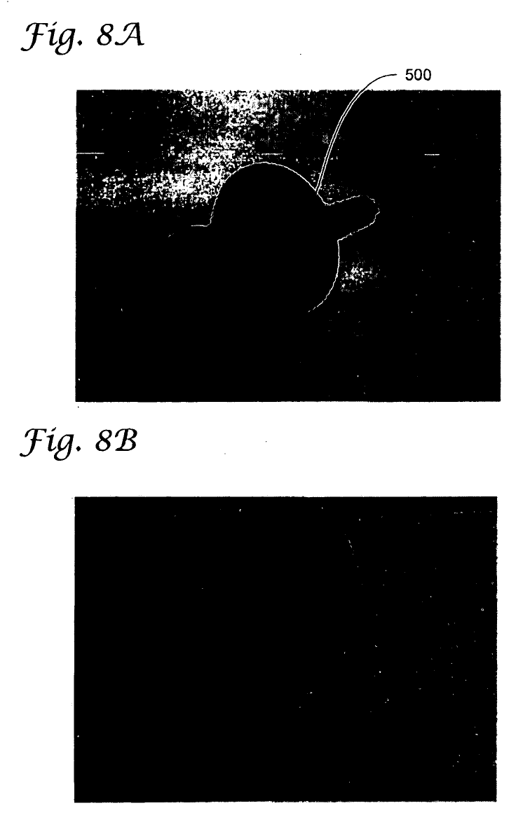

The tip 201 of the collector 150 is preferably inflated using air during transfer. When the tip 201 is made from a thermoplastic elastomer alloy such as Versaflex® CL30, the elastomer allows uniform expansion of the tip during inflation. During inflation, the tip region 210 and the transition section 212 substantially go away (see FIG. 8B) so that the cell clusters on the tip region 210 and transition section 212 end up generally on a common plane for subsequent transfer of cell clusters to the receiving structure. This helps to maintain the spatial relationship of the cells in the cell clusters.

After transfer, the tip 201 can be removed from the tube 200 and put into a container with preservative to preserve remaining cell clusters on the tip 201. The tube 200 can then be discarded or connected to a new tip 201 for further collections. If the tip 201 does not need to be preserved, the tip 201 can be discarded.

FIG. 8A shows a tip 201 of a collector with colored marker 500 on the tip simulating collected transition zone cell clusters. FIG. 8B shows the tip 201 inflated, showing how the colored marker 500 simulating the cell clusters is faint but still visible.

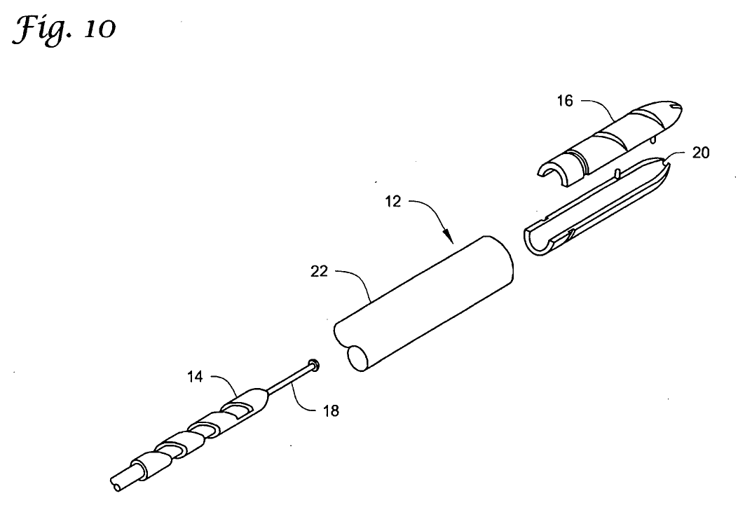

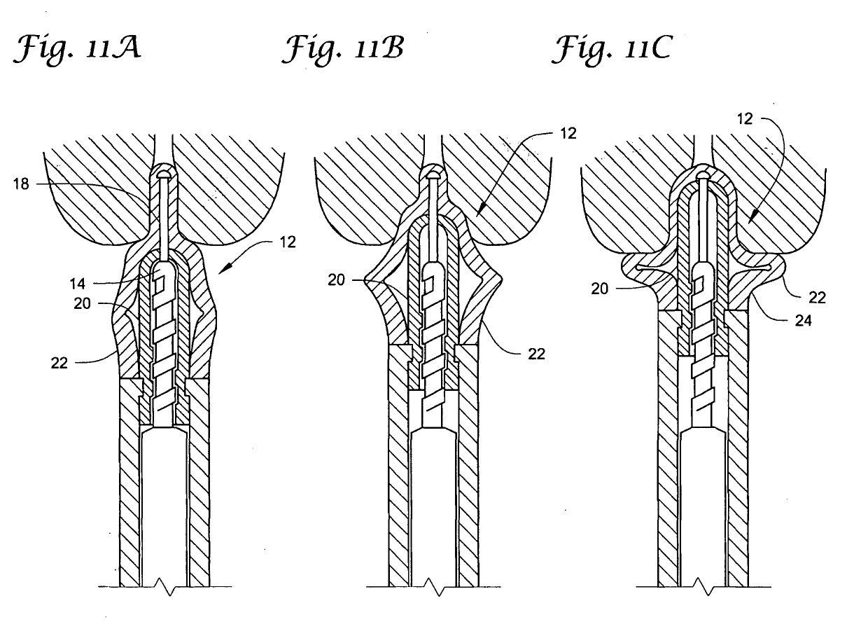

With reference now to FIGS. 10 and 11A-C, another embodiment of a cervical cell collector 10 for collecting cells in a uterine cervical canal is illustrated. In this example, the cervical cell collector 10 is comprised of an assembly that includes a flexible cell sampling region 12 and abutting rigid pusher 22 within which is contained a second assembly consisting of a tip expander 16 rotatably mounted on a rigid core element 14 with one set of features 31 of the tip expander engaging corresponding actuating features 32 of the core element 14 and a second set of features 33 engaging mating features of the pusher 34. The actuating features 32 of the core element 14 are configured, by way of example, as a screw thread having a suitable pitch. A stylette 18 attached to the core element 14 passes through an opening 20 in the tip expander 16.

The cell sampling region 12 can be a resiliently flexible structure that is made of a suitable elastomeric material such as microporous polyvinyl acetate, thermoplastic elastomer, nitrile rubber, nitrile foam, urethane foam, silicone rubber, latex rubber, polyurethane or any material having suitable low durometer, high percent elongation and surface qualities.

As suggested by FIGS. 11A, 11B, and 11C, the cervical cell collector can transition between an extended state (FIG. 11A); an intermediate state (FIG. 11B); and a collapsed state (FIG. 11C). The clinician guides the tip of the cervical cell collector 10 in its extended state into the cervical canal to the desired depth (indicated as the tip depth) as shown in FIG. 11A. In this state, the pusher 22 is retracted and the cell sampling member 12 is approximately conformal to the exterior surface of the tip expander 16. Once the clinician has properly positioned the tip of the cervical cell collector 10 in the cervical canal, the pusher is advanced toward the cervical os while the core element 14 and stylette 18 remain stationary. As features 31 and 34 of the pusher 22 are engaged with corresponding features 32 and 33 of core element 14 and tip expander 16, respectively, advancing the pusher 22 causes the tip expander 15 to likewise move toward the os and to rotate around stationary core element 14. Concurrently, advancement of pusher 22 applies a compressive force to the cell sampling member 12 thereby causing it to deform radially outward against the exterior portion of the cervical os as is shown in FIGS. 11B and 11C. Advancement of the tip expander 16 into the tip of the cell sampling member 12 causes the diameter of tip of the cell sampling member 12 to increase, thereby pressing the exterior surface of the cell sampling member 12 against the walls of the cervical canal. The rotary motion of the tip expander 16 relative to the interior surface of the cell sampling member 12 facilitates entry of the tip expander into and, thereby, the expansion of the cell sampling member.

Contact and rotation of the cell sampling member 12 against the surfaces of the cervical os and cervical canal causes exfoliated cervical cells to adhere to the exterior surface of the cell sampling member. Retraction of the pusher 22 withdraws the tip expander 16 from the tip of the cell sampling member 12, thus allowing the cell sampling member to return to its initial extended state. The cervical cell collector 10 may then be removed from the cervical canal 100 and vagina and the cells collected on the surface of the cell sampling member prepared for analysis.

Cell clusters of the ecto- and endocervices are collected using a collector with the characteristics described above. The sample may be collected by a physician or health care worker. Alternately, it should be possible to train women to collect their own samples.

While the invention has been described in conjunction with a preferred embodiment, it will be obvious to one skilled in the art that other objects and refinements of the present invention may be made with the present invention within the purview and scope of the present invention.

The invention, in its various aspects and disclosed forms, is well adapted to the attainment of the stated objects and advantages of others. The disclosed details are not to be taken as limitations on the invention.

1. A cervical cell collector comprising:
a main body;
a resilient surface disposed proximate one end of the main body, the resilient surface having a contact texture being suitable for collecting clusters of cells from an ectocervical region and an endocervical region of a cervix, the resilient surface being made of a material that allows uniform expansion of the resilient surface, and the resilient surface may be rotatable; and
a band to connect the resilient surface to the main body,
wherein when the resilient surface is expanded and rotated, the contact texture of the resilient surface enhances the collection of clusters of cells from the ectocervical and endocervical regions by the resilient surface.

2. The cervical cell collector of claim 1, wherein the resilient surface being a tip portion disposed at one end of the main body.

3. The cervical cell collector of claim 2, wherein the tip portion is detachably connected to the main body.

4. The cervical cell collector of claim 1, wherein the material of the resilient surface comprises a thermoplastic elastomer alloy.

5. The cervical cell collector of claim 1, wherein the contact texture comprises MT-11010.

6. The cervical cell collector of claim 1, further comprising at least one orientation indicator disposed proximate the resilient surface.

7. The cervical cell collector of claim 1, wherein during collection the resilient surface includes an enlarged shoulder, a tip region, and a transition section between the tip region and the enlarged shoulder, and wherein the resilient surface is expandable to an extent that the tip region and the transition section substantially disappear and cell clusters on the tip region and the transition section end up on generally a common plane.

8-11. (canceled)

12. A method for collecting cervical cells, the method comprising:
contacting clusters of cells at an ectocervical region and an endocervical region of a cervix, the clusters of cells being contacted with a resilient surface of a collector, the resilient surface having a contact texture being suitable for collecting clusters of cells from the ectocervical and endocervical regions;
expanding the resilient surface of the collector; and
rotating the resilient surface with respect to the ectocervical and endocervical regions;
wherein when the resilient surface is expanded and rotated, the contact texture of the resilient surface enhances the collection of clusters of cells from the ectocervical and endocervical regions by the resilient surface,
wherein the expanding step includes mechanically expanding the collector.

13. (canceled)

14. The method of claim 12, comprising manually rotating the resilient surface.

15. The method of claim 12, comprising mechanically rotating the resilient surface.

16. (canceled)

17. The method of claim 12, comprising rotating the resilient surface after expanding the resilient surface.

18. The cervical cell collector of claim 1, wherein the resilient surface is a single layer structure.

19. The method of claim 12, wherein the collector is mechanically expanded using a ring.

20. The method of claim 20, wherein the ring is a shoulder.
